(19) United States
(12) Patent Application Publication
(10) Pub. No.: US 2018/0280349 A1
(43) Pub. Date: Oct. 4, 2018

(54) METHODS OF TREATING CYSTIC FIBROSIS IN PATIENTS WITH RESIDUAL FUNCTION MUTATIONS

(71) Applicants: Fredrick F. Van Goor, San Diego, CA (US); Edward Ingenito, Quincy, MA (US)

(72) Inventors: Fredrick F. Van Goor, San Diego, CA (US); Edward Ingenito, Quincy, MA (US)

(73) Assignee: Vertex Pharmaceuticals Incorporated

(21) Appl. No.: 15/937,564

(22) Filed: Mar. 27, 2018

Related U.S. Application Data
(60) Provisional application No. 62/477,937, filed on Mar. 28, 2017.

(51) Int. Cl. A61K 31/404; A61P 11/00; A61K 31/47; A61K 45/06

(52) U.S. Cl. CPC A61K 31/404; A61K 45/06; A61K 31/47; A61P 11/00

(57) ABSTRACT

Modulators of Cystic Fibrosis Transmembrane Conductance Regulator (CFTR), their pharmaceutical compositions, and methods of treating cystic fibrosis in patients with residual function mutations.

This application describes modulators of Cystic Fibrosis Transmembrane Conductance Regulator (CFTR), their pharmaceutical compositions, and methods of treating cystic fibrosis in patients with residual function mutations.

Cystic fibrosis (CF) is a recessive genetic disease that affects approximately 70,000 children and adults worldwide. Despite progress in the treatment of CF, there is no cure.

In patients with CF, mutations in CFTR endogenously expressed in respiratory epithelia lead to reduced apical anion secretion, causing an imbalance in ion and fluid transport. The resulting decrease in anion transport contributes to enhanced mucus accumulation in the lung and accompanying microbial infections that ultimately cause death in CF patients. In addition to respiratory disease, CF patients typically suffer from gastrointestinal problems and pancreatic insufficiency that, if left untreated, result in death. In addition, the majority of males with cystic fibrosis are infertile, and fertility is reduced among females with cystic fibrosis.

Sequence analysis of the CFTR gene has revealed a variety of disease-causing mutations (Cutting, G. R. et al. (1990) Nature 346:366-369; Dean, M. et al, (1990) Cell 61:863:870; and Kerem, B-S. et al. (1989) Science 245:1073-1080; Kerem, B-S et al. (1990) Proc. Natl. Acad. Sci. USA 87:8447-8451). To date, greater than 2000 disease-causing mutations in the CF gene have been identified and about 242 of these mutations are known to cause cystic fibrosis. The most prevalent protein mutation is a deletion of phenylalanine at position 508 of the CFTR amino acid sequence, and is commonly referred to as F508del protein mutation. This mutation occurs in approximately 70% of the cases of cystic fibrosis and is associated with severe disease. Other commonly occurring mutations include 621+1G→T, 1717-1G→A, 2789+5G→A, 3120+1G→A, 3849+10kbC→T, A455E, G542X, G551D, N1303K, R117H, R553X, and W1282X.

CFTR is a cAMP/ATP-mediated anion channel that is expressed in a variety of cell types, including absorptive and secretory epithelia cells, where it regulates anion flux across the membrane, as well as the activity of other ion channels and proteins. In epithelial cells, normal functioning of CFTR is critical for the maintenance of electrolyte transport throughout the body, including respiratory and digestive tissue. CFTR is composed of approximately 1480 amino acids that encode a protein which is made up of a tandem repeat of transmembrane domains, each containing six transmembrane helices and a nucleotide binding domain. The two transmembrane domains are linked by a large, polar, regulatory (R)-domain with multiple phosphorylation sites that regulate channel activity and cellular trafficking.

Chloride absorption takes place by the coordinated activity of ENaC and CFTR present on the apical membrane and the Na⁺—K⁺-ATPase pump and Cl— channels expressed on the basolateral surface of the cell. Secondary active transport of chloride from the luminal side leads to the accumulation of intracellular chloride, which can then passively leave the cell via Cl⁻ channels, resulting in a vectorial transport. Arrangement of Na⁺/2Cl⁻/K⁺ co-transporter, Nat⁺—K⁺-ATPase pump and the basolateral membrane K⁺ channels on the basolateral surface and CFTR on the luminal side coordinate the secretion of chloride via CFTR on the luminal side. Because water is probably never actively transported itself, its flow across epithelia depends on tiny transepithelial osmotic gradients generated by the bulk flow of sodium and chloride.

The clinical impact of any CFTR mutation is believed to be related to the amount of total CFTR ion transport activity. A CFTR mutation may affect the CFTR quantity, i.e., the number of CFTR channels at the cell surface, or it may impact CFTR function, i.e., the functional ability of each channel to open and transport ions. Mutations affecting CFTR quantity include mutations that cause defective synthesis (Class I defect), mutations that cause defective processing and trafficking (Class II defect), mutations that cause reduced synthesis of CFTR (Class V defect), and mutations that reduce the surface stability of CFTR (Class VI defect). Mutations that affect CFTR function include mutations that cause defective gating (Class III defect) and mutations that cause defective conductance (Class IV defect).

Some CFTR mutations reduce CFTR protein quantity or function to such an extent that there is little to no total CFTR activity. Other mutations result only in reduced protein quantity or function at the cell surface which can produce partial CFTR activity. These mutations are called residual function mutations. For example, some CFTR mutations that cause defective mRNA splicing, such as 2789+5G→A and E831X, result in reduced protein synthesis, but deliver some functional CFTR to the surface of the cell to provide residual function. Other CFTR mutations that reduce conductance and/or gating, such as R117H, result in a normal quantity of CFTR channels at the surface of the cell, but the functional level is low, resulting in residual function. Some mutations, such as F508del, result in multiple CFTR protein defects.

Both CFTR alleles play a role in determining phenotype of disease severity. Common residual function mutations include E56K, P67L, R74W, D110E, D110H, R117C, R117H, G178R, E193K, L206W, R347H, R352Q, A455E, S549N, S549R, G551D, G551S, D579G, 711+3A→G, E831X, S945L, S977F, F1052V, K1060T, A1067T, R1070W, F1074L, D1152H, G1244E, S1251N, S1255P, D1270N, G1349D, 2789+5G→A, 3272-26A-→G, and 3849+10kbC→T. Patients with residual function mutations may experience the symptoms of CFTR-mediated diseases later in life and symptoms may be less severe than in patients with other mutations. Patients with CFTR residual function mutations tend to have higher rates of pancreatic sufficiency, less elevated sweat chloride levels, and less severe pulmonary disease than patients with other mutations. However, patients with a residual function mutation generally have progressive lung function decline and other complications of CF that may still lead to a severe disease stage and cause premature death. The life expectancy and quality of life for CFTR residual function mutation patients is well below that of persons without cystic fibrosis. Accordingly, there is a need for treatment of CFTR-mediated diseases, particularly in those patients with residual function mutations.

Thus, one aspect of the invention provides methods of modulating CFTR-mediated diseases, particularly cystic fibrosis, in patients with residual function mutations by administering (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound I) or a pharmaceutically acceptable salt thereof and administering N-(2,4-di-tert-butyl-5-hydroxyphenyl)-4-oxo-1,4-dihydroquinoline-3-carboxamide (Compound II) or N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound II-d) or a pharmaceutically acceptable salt of one of Compound II or II-d, wherein the compounds are administered simultaneously, serially, or sequentially, in a single composition, or in separate compositions. Pharmaceutical compositions comprising (1) Compound I or a pharmaceutically acceptable salt thereof, (2) Compound II or II-d or a pharmaceutically acceptable salt of either, and (3) both Compound I and Compound II or II-d or a pharmaceutically acceptable salt of any of the foregoing, may include at least one additional active pharmaceutical ingredient and may include at least one carrier. In some embodiments, the CFTR residual function mutation is selected from E56K, P67L, R74W, D110E, D110H, R117C, R117H, G178R, E193K, L206W, R347H, R352Q, A455E, S549N, S549R, G551D, G551S, D579G, 711+3A→G, E831X, S945L, S977F, F1052V, K1060T, A1067T, R1070W, F1074L, D1152H, G1244E, S1251N, S1255P, D1270N, G1349D, 2789+5G→A, 3272-26A→G, and 3849+10kbC→T.

In some embodiments, the residual function mutation is a splice mutation selected from 2789+5G→A, 3272-26A→G, 3849+10kbC→T, 711+3A→G, and E831X. In some embodiments, the splice mutation is E831X.

In some embodiments, the CFTR mutation is E831X.

In some embodiments, the CFTR residual function mutation is a missense mutation selected from D579G, D110H, D1152H, A455E, L206W, P67L, R1070W, R117C, R347H, R352Q, S945L, and S977F.

In some embodiments, the CFTR residual function mutation is selected from R117H, G178R, S549N, S549R, G551D, G551S, G1244E, S1251N, and G1349D.

In one embodiment, the patient is heterozygous for at least one residual function mutation on one allele and a second CFTR gene mutation on the other allele. In another embodiment, the patient is heterozygous for a E831X mutation on one allele and a F508del mutation on the other allele. In one embodiment, the patient has at least one E831X mutation.

### BRIEF DESCRIPTION OF THE DRAWINGS

FIG. 1 identifies representative Ma mutations.

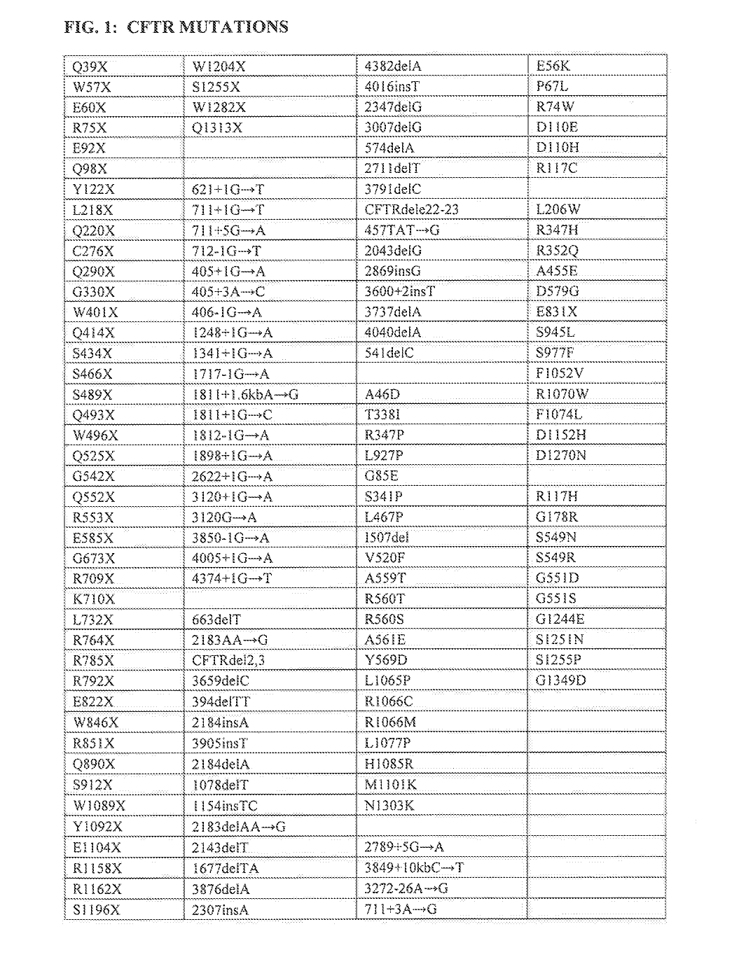

FIG. 2 shows the absolute change in lung function over time for patients dosed with 100 mg Compound I every 24 hours arid 150 mg Compound II or II-d every 12 hours and patients dosed with 150 mg Compound II or II-d every 12 hours after 8 weeks of treatment.

### DEFINITIONS

As used herein, “CFTR” means cystic fibrosis transmembrane conductance regulator.

As used herein, “mutations” can refer to mutations in the CFTR gene or the CFTR protein. A “CFTR gene mutation” refers to a mutation in the CFTR gene, and a “CFTR protein mutation” refers to a mutation in the CFTR protein. A genetic defect or mutation, or a change in the nucleotides in a gene in general results in a mutation in the CFTR protein translated from that gene.

A “residual function mutation” as used herein, refers to a mutation in the CFTR gene that results in reduced CFTR protein quantity or function of the protein at the cell surthce, CFTR gene mutations known to result in a residual function phenotype include, in some embodiments, CFTR residual function mutations selected from E56K, P67L, R74W, D110E, D110H, R117C, R117H, G178R, E193K, L206W, R347H, R352Q, A455E, S549N, S549R, G551D, G551S, D579G, 711+3A→G, E831X, S945L, S977F, F1052V, K1060T, A1067T, R1070W, F1074L, D1152H, G1244E, S1251N, S1255P, D1270N, G1349D, 2789+5G→A, 3272-26A→G, and 3849+10kbC→T.

Residual Function in CF is determined clinically based on population characteristics such as lower sweat chloride levels and incidence of pancreatic sufficiency. Residual function may be indicative of the presence of a CFTR mutation that results in some functional CFTR protein at the cell surface leading to residual CFTR ion transport activity.

Residual CFTR function can be characterized at the cellular (in vitro) level using cell-based assays, such as an FRT assay (Van Goor, F. et al, (2009) PNAS Vol. 106, No. 44, 18825-18830; and Van Goor, F. et al. (2011) PNAS Vol. 108, No. 46, 18843-18846) to measure the amount of chloride transport through the mutated CFTR channels. Residual function mutations result in a reduction hut not complete elimination of CFTR dependent ion transport. In some embodiments, residual function mutations result in at least about 10% reduction of CFTR activity in an FRT assay. In some embodiments, the residual function mutations result in up to about 90% reduction in CFTR activity in an FRT assay.

These residual function patients have variable disease with some patients showing a delayed decline in lung function or age of diagnosis compared to patients with more serious CFTR mutations such as, for example, homozygous for F508del.

While patients carrying CFTR mutations associated with residual function activity demonstrate variability in their clinical phenotype, which may include delayed disease progression, patients with residual function mutations develop chronic pulmonary disease, experience pulmonary exacerbations, have an increasing frequency of hospitalizations over the course of their lifespan, and have a markedly reduced median life expectancy compared with the general population.

As used herein, a patient who is “homozygous” for a particular gene mutation has the same mutation on each allele. The term “heterozygous” as used herein, refers to a patient having a particular gene mutation on one allele, and a different mutation or no mutation on the other allele. Patients that may benefit from the methods of treatment of the invention and from pharmaceutical compositions described herein for use in treating CFTR-mediated diseases include patients who have homozygous or heterozygous mutations on the CFTR gene, but also have a residual function phenotype.

As used herein, the term “modulator” refers to a compound that alters or increases the activity of a biological compound such as a protein. For example, a CFTR modulator is a compound that generally increases the activity of CFTR. The increase in activity resulting from a CFTR modulator includes but is not limited to compounds that correct, potentiate, stabilize and/or amplify CFTR.

As used herein, the term “CFTR corrector” refers to a compound that increases the amount of functional CFTR protein at the cell surface, resulting in enhanced ion transport. Compound I disclosed herein is a CFTR corrector.

As used herein, the term “CFTR potentiator” refers to a compound that increases the channel activity of CFTR protein located at the cell surface, resulting in enhanced ion transport. Compound II and as disclosed herein are CFTR potentiators.

As used herein, the term “active pharmaceutical ingredient” or “API” refers to a biologically active compound.

As used herein, the term “amorphous” refers to a solid material having no long-range order in the position of its molecules. Amorphous solids are generally supercooled liquids in which the molecules are arranged in a random manner so that there is no well-defined arrangement, e.g., molecular packing, and no long-range order. Amorphous solids are generally isotropic, i.e. exhibit similar properties in all directions and do not have definite melting points. For example, an amorphous material is a solid material having no sharp characteristic crystalline peak(s) in its X-ray power diffraction (XRPD) pattern (i.e., is not crystalline as determined by XRPD). Instead, one or several broad peaks (e.g., halos) appear in its XRPD pattern. Broad peaks are characteristic of an amorphous solid. See, US 2004/0006237 for a comparison of XRPDs of an amorphous material and crystalline material.

As used herein, the term “substantially amorphous” refers to a solid material having little or no long-range order in the position of its molecules. For example, substantially amorphous materials have less than 15% crystallinity (e.g., less than 10% crystallinity or less than 5% crystallinity). It is also noted that the term “substantially amorphous” includes the descriptor, “amorphous,” which refers to materials having no (0%) crystallinity.

As used herein, the term “dispersion” refers to a disperse system in which one substance, the dispersed phase, is distributed, in discrete units, throughout a second substance (the continuous phase or vehicle). The size of the dispersed phase can vary considerably (e.g. colloidal particles of nanometer dimension, to multiple microns in size). In general, the dispersed phases can be solids, liquids, or gases. In the case of a solid dispersion, the dispersed and continuous phases are both solids. In pharmaceutical applications, a solid dispersion can include a crystalline drug (dispersed phase) in an amorphous polymer (continuous phase); or alternatively, an amorphous drug (dispersed phase) in an amorphous polymer (continuous phase). In some embodiments, a solid dispersion includes the polymer constituting the dispersed phase, and the drug constitute the continuous phase. Or, a solid dispersion includes the drug constituting the dispersed phase, and the polymer constituting the continuous phase.

The term “patient” or “subject” is used interchangeably and refers to an animal including humans.

The terms “effective dose” or “effective amount” are used interchangeably herein and refer to that amount of a compound that produces the desired effect for which it is administered (e,g., the treatment of CF, improvement in CF or a symptom of CF, or lessening the severity of CF or a symptom of CF). The exact amount of an effective dose will depend on the purpose of the treatment, and the patient, and will be ascertainable by one skilled in the art using known techniques (see, e.g., Lloyd (1999) The Art, Science and Technology of Pharmaceutical Compounding).

As used herein, the terms “treatment,” “treating,” and the like generally mean the improvement of CF or its symptoms or lessening the severity of CF or its symptoms in a subject. “Treatment,” as used herein, includes, but is not limited to, the following: increased growth of the subject, increased weight gain, reduction of mucus in the lungs, improved pancreatic and, or liver function, reduction of chest infections, increase in FEV₁ (forced expiratory volume in one second), decreases in sweat chloride, reductions in exacerbations, increased life span, decreased progression of disease, and/or reductions in coughing or shortness of breath. Improvements in or lessening the severity of any of these symptoms can be readily assessed according to standard methods and techniques known in the art.

As used herein, the term “in combination with” when referring to two or more compounds, agents, or additional active pharmaceutical ingredients, means the administration of two or more compounds, agents, or active pharmaceutical ingredients to the patient prior to, concurrent with, or subsequent to each other. The terms “about” and “approximately,” when used in connection with doses, amounts, or weight percent of ingredients of a composition or a dosage form, include the value of a specified dose, amount, or weight percent, or a range of the dose, amount, or weight percent that is recognized by one of ordinary skill in the art to provide a pharmacological effeet equivalent to that obtained from the specified dose, amount, or weight percent.

### Compounds I, II, and II-d

The chemical structure of Compound I is:

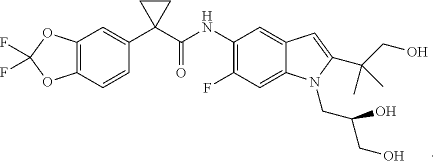 [SMILES: CC(C)(CO)c1cc2cc(NC(=O)C3(c4ccc5c(c4)OC(F)(F)O5)CC3)c(F)cc2n1C[C@@H](O)CO]

The IUPAC name for Compound I is (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide. The generic name for Compound I is tezacaftor.

The chemical structure of Compound II is:

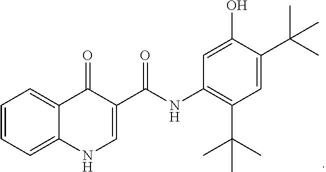 [SMILES: CC(C)(C)c1cc(C(C)(C)C)c(NC(=O)c2c[nH]c3ccccc3c2=O)cc1O]

The IUPAC name for Compound II is N-(2,4-di-tert-butyl-5-hydroxyphenyl)-4-oxo-1,4-dihydroquinoline-3-carboxamide. The generic name for Compound II is ivacaftor.

Compound I and pharmaceutically acceptable salts thereof and methods of making Compound I and its pharmaceutically acceptable salts are described in U.S. Pat. No. 7,645,789 at Col. 464-468, and in U.S. Pat. No.9,035,072 at Col. 42-55, both incorporated herein by reference. Compound II and pharmaceutically acceptable salts thereof and methods of making Compound II and its pharmaceutically acceptable salts are described in U.S. Pat. No. 7,495,103 at Col. 106, 107, 153-155, 221, 226, 256, and 269 and in U.S. Pat. No. 8,476,442 at Col. 56-58 and 91-98; both incorporated herein by reference.

In some embodiments of the invention either or both of Compound I and Compound II may have one or more isotopically enriched atoms. For example, one or more hydrogens in Compound I and/or Compound II may optionally be replaced by deuterium or tritium, or carbon may optionally be replaced by ¹³C- or ¹⁴C-enriched carbon. Such compounds are useful, for example, as analytical tools or probes in biological assays, or as therapeutic agents.

Deuterated analogs of Compound I for use in treating CFTR-mediated diseases are disclosed in PCT Publication No. WO 2016/160945, incorporated herein by reference. Deuterated analogs of Compound II for use in treating CFTR-mediated diseases are disclosed in U.S. Pat. No. 8,865,902, incorporated herein by reference.

In some embodiments, the deuterated analog of Compound II is:

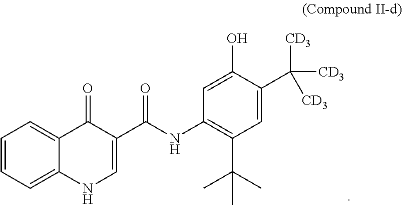 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

### Pharmaceutically Acceptable Salts

A “pharmaceutically acceptable salt” as used herein refers to any salt or salt of an ester of a compound of this disclosure that, upon administration to a recipient, is capable of providing, either directly or indirectly, a compound of this disclosure. Pharmaceutically acceptable salts are well known in the art. For example, S. M. Berge, et al. describe pharmaceutically acceptable salts in detail in J. Pharmaceutical Sciences, 1977, 66, 1-19, such as those found in Table 1:

TABLE 1
FDA-Approved Commercially Marketed Salts
Anion

Acetate
Aluminum
Benzenesulfonate
Benzathine
Benzoate
Bicarbonate
Bitartrate
Bromide
Calcium
Calcium edetate
Camsylate
Carbonate
Chloride
Choline
Citrate
Diethanolamine
Dihydrochloride
Edetate
Edisylate
Estolate
Esylate
Ethylenediamine
Fumarate
Gluceptate
Gluconate
Glutamate
Glycollylarsanilate
Hexylresorcinate
Hydrabamine
Hydrobromide
Hydrochloride
Hydroxynaphthoate
Iodide
Isethionate
Lactate
Lactobionate
Lithium
Magnesium
Malate
Maleate
Mandelate
Meglumine
Mesylate
Methylbromide
Methylnitrate
Methylsulfate
Mucate
Napsylate
Nitrate
Pamoate (Embonate)
Pantothenate
Phosphate/diphosphate
Polygalacturonate
Potassium
Potassium
Salicylate
Sodium
Stearate
Subacetate
Succinate
Sulfate
Tannate
Tartrate
Teociate
Triethiodide
Zinc

Pharmaceutically acceptable salts of Compound I and Compound II or II-d include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄ alkyl)₄ salts. The quaternization of any basic nitrogen-containing groups of Compound I and/or Compound II or II-d are also envisioned, Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, ammonium, quaternary ammonium, and amine cations formed using counterions such as halide, hydroxide, carboxylate, sulfate, phosphate, nitrate, lower alkyl sulfonate and aryl sulfonate. Other representative pharmaceutically acceptable salts include besylate and glucosamine salts.

### Pharmaceutical Compositions

A pharmaceutical composition for use in the methods of the invention comprise, in addition to Compound I and Compound II or II-d or a pharmaceutically acceptable salt of any of the foregoing, one or more of a vehicle, adjuvant, or carrier, such as a filler, a disintegrant, a surfactant, a binder, a lubricant, or combinations thereof.

Compositions comprising Compound I and Compound II are described in United States Patent Application Publication US 2015/0320736 A1 at pages 64, 65, 67, and 68, incorporated herein by reference.

In some embodiments, the methods of the invention employ a pharmaceutical composition comprising Compound I or a pharmaceutically acceptable salt thereof, and a pharmaceutically acceptable carrier. In some embodiments, the methods of the invention employ a pharmaceutical composition comprising Compound II or II-d or a pharmaceutically acceptable salt thereof, and a pharmaceutically acceptable carrier. in some embodiments, the methods of the invention employ a pharmaceutical composition comprising both Compound I and Compound II or II-d or a pharmaceutically acceptable salt of one or both of Compound I and. Compound II or II-d, and a pharmaceutically acceptable carrier.

A pharmaceutical composition disclosed herein additionally may comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington: The Science and Practice of Pharmacy, 21st edition, 2005, ed. D. B. Troy, Lippincott Williams & Wilkins, Philadelphia, and Encyclopedia of Pharmaceutical Technology, eds. J. Swarbrick and J. C. Boylan, 1988-1999, Marcel Dekker, New York disclose various carriers used in formulating pharmaceutically acceptable compositions and known techniques for the preparation thereof. Except insofar as any conventional carrier medium is incompatible with the compounds of this disclosure, such as by producing any undesirable biological effect or otherwise interacting in a deleterious manner with any other component(s) of the pharmaceutically acceptable composition, its use is contemplated to be within the scope of this disclosure. Some examples of materials which can serve as pharmaceutically acceptable carriers include, but are not limited to, ion exchangers, alumina, aluminum stearate, lecithin, serum proteins such as human serum albumin, buffer substances such as phosphates, glycine, sorbic acid, or potassium sorbate, partial glyceride mixtures of saturated vegetable fatty acids, water, salts or electrolytes such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate, powdered tragacanth, malt, gelatin, talc, excipients such as cocoa butter and suppository waxes, oils such as peanut oil, cottonseed oil, safflower oil, sesame oil, olive oil, corn oil and soybean oil, glycols such as propylene glycol or polyethylene glycol, esters such as ethyl oleate and ethyl laurate, agar, buffering agents such as magnesium hydroxide and aluminum hydroxide, alginic acid, pyrogen-free water, isotonic saline, Ringer's solution, ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives, and antioxidants can also be present in the composition, according to the judgment of the formulator.

A listing of exemplary embodiments includes:
1. Use of (R)-1-(2,2-difluorobenzo[d][1,3]dioxol -5-yl)-N-(1-(2,3-dihydroxypropyl)-6-fluoro-2-(1-hydroxy-2-methylpropan-2-yl)-1H-indol -5-yl)cyclopropanecarboxamide (Compound I):

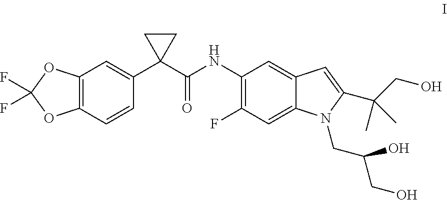 [SMILES: CC(C)(CO)c1cc2cc(NC(=O)C3(c4ccc5c(c4)OC(F)(F)O5)CC3)c(F)cc2n1C[C@@H](O)CO]

or a pharmaceutically acceptable salt thereof and N-(2,4-di-tert-butyl-5-hydroxyphenyl)-4-oxo-1,4-dihydroquinoline-3-carboxamide (Compound II):

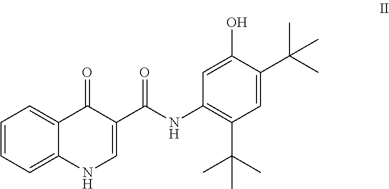 [SMILES: CC(C)(C)c1cc(C(C)(C)C)c(NC(=O)c2c[nH]c3ccccc3c2=O)cc1O]

or N-(2-(tert-butyl)-5-hydroxy-4-(2-(methyl-d3)propan-2-yl-1,1,1,3,3-d6)phenyl)-4-oxo-1,4-dihydroquinoline-3-carboxamide (Compound II-d):

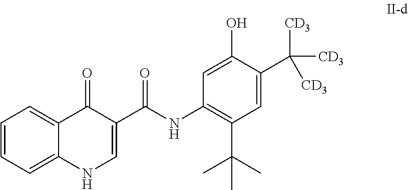 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

or a pharmaceutically acceptable salt of one of Compound II and Compound II-d, for treating cystic fibrosis in a patient, wherein the patient has at least one E831X cystic fibrosis transmembrane conductance regulator (CFTR) mutation,
2. The use according to embodiment 1, comprising administering to the patient an effective amount of N-(2,4-di-tert-butyl-5 -hydroxyphenyl)-4-oxo-1,4-dihydroquinoline-3-carboxamide (Compound II):

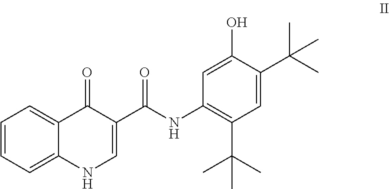 [SMILES: CC(C)(C)c1cc(C(C)(C)C)c(NC(=O)c2c[nH]c3ccccc3c2=O)cc1O]

or a pharmaceutically acceptable salt thereof.
3. The use according to embodiment 1, comprising administering to the patient an effect amount of (N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound II-d):

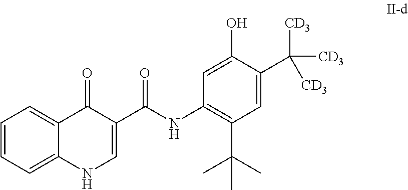 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]

or a pharmaceutically acceptable salt thereof.
4, The use according to embodiment 1, wherein the patient has a second CFTR mutation that is F508del.
5, The use according to embodiment 2, Wherein the patient has a second CFTR mutation that is F508del.
6. The use according to embodiment 3, wherein the patient has a second CFTR mutation that is F508del.
7, The use according to embodiment 1, comprising administering a pharmaceutical composition of Compound I or a pharmaceutically acceptable salt thereof concurrently with, prior to, or subsequent to a pharmaceutical composition comprising Compound II or II-d or a pharmaceutically acceptable salt thereof.
8. The use according to embodiment 7, further comprising administering a pharmaceutical composition comprising at least one additional active pharmaceutical ingredient.
9. The use according to embodiment 8, wherein the at least one additional active pharmaceutical ingredient is administered simultaneously, sequentially, in a single composition, or as one or more separate compositions.
10. The use according to embodiment 8, wherein the at least one additional active pharmaceutical ingredient is a CFTR modulator.
11. The use according to embodiment 2, further comprising administering a pharmaceutical composition comprising the at least one additional active pharmaceutical ingredient, wherein the at least one additional active pharmaceutical ingredient is a CFTR modulator.
12. The use according to embodiment 3, further comprising administering a pharmaceutical composition comprising the at least one additional active pharmaceutical ingredient, wherein the at least one additional active pharmaceutical ingredient is a CFTR modulator.
13. The use according to embodiment 8, wherein the CFTR modulator is selected from a CFTR corrector and a CFTR potentiator.
14. The use according to embodiment 1, wherein the patient exhibits residual CFTR activity in the apical membrane of respiratory and non-respiratory epithelia.
15. The use according to embodiment 1, wherein the patient exhibits little to no CFTR activity in the apical membrane of respiratory epithelia.

### Methods of Treatment

Compound II (ivacaftor) has been approved by the U.S. Food and Drug Administration for treating people with cystic fibrosis. In February 2018 the FDA approved Compound I/Compound II (tezacaftor/ivacaftor) for use in treating cystic fibrosis in patients. In some embodiments, the patients have a mutation selected from E56K, P67L, R74W, D110E, D110H, R117C, E193K, L206W, R347H, R352Q, A455E, D579G, 711+3A→G, E831X, S945L, S977F, F1052V, K1060T, A1067T, R1070W, F1074L, D1152H, D1270N, 2789+5G→A, 3272-26A→G, and 3849+10kbC→T.

One aspect of the invention provides a method of modulating a CFTR-mediated disease in a patient with a CFTR residual function mutation, by administering Compound I and Compound II or II-d. In some embodiments, the residual function mutation results in the patient suffering from cystic fibrosis or symptoms thereof. In certain embodiments, Compound I or a pharmaceutically acceptable salt thereof is administered in combination with Compound II or II-d or a pharmaceutically acceptable salt thereof as separate compositions. In other embodiments, Compound I or a pharmaceutically acceptable salt thereof is administered together with Compound II or II-d or a pharmaceutically acceptable salt thereof in a single composition.

Thus, one aspect of the invention provides a method of modulating CFTR activity in a patient with a residual function mutation resulting in cystic fibrosis, by administering a pharmaceutical composition comprising Compound I or a pharmaceutically acceptable salt thereof, which may include a pharmaceutically acceptable carrier and simultaneously or sequentially administering a pharmaceutical composition comprising Compound II or II-d or a pharmaceutically acceptable salt thereof, which may include a pharmaceutically acceptable carrier. In some embodiments, the method of modulating CFTR activity in a patient having a residual function mutation, by administering a pharmaceutical composition comprising a pharmaceutical composition comprising both Compound I and Compound II or II-d or a pharmaceutically acceptable salt of either or both Compound I and Compound II or II-d, may include at least one additional active pharmaceutical ingredient and may include at least one carrier.

Another aspect of the invention provides a method of modulating a CFTR-mediated disease in a patient with a residual function mutation, by administering Compound I or a pharmaceutically acceptable salt thereof, Compound II or II-d or a pharmaceutically acceptable salt thereof, and at least one additional active pharmaceutical ingredient, simultaneously, sequentially, in a single composition, or as one or more separate compositions. In one embodiment, the at least one active pharmaceutical ingredient is a CFTR modulator. In one embodiment, the at least one active pharmaceutical ingredient is a CFTR corrector. In one embodiment, the at least one active pharmaceutical ingredient is a CFTR potentiator. In some embodiments, the CFTR potentiator is Compound II-d.

One aspect of the invention provides a method of treating or lessening the severity of cystic fibrosis in a patient, comprising administering to the patient an effective amount of Compound I or pharmaceutically acceptable salt thereof and Compound II or II-d, or a pharmaceutically acceptable salt of either. Another aspect of the invention provides a method of treating cystic fibrosis in a patient, comprising administering to the patient an effective amount of Compound I or pharmaceutically acceptable salt thereof and Compound II or II-d, or a pharmaceutically acceptable salt of either. Another aspect provides the method of lessening the severity of cystic fibrosis in a patient, comprising the step of administering to the patient an effective amount of Compound I or pharmaceutically acceptable salt thereof and Compound II or II-d, or a pharmaceutically acceptable salt of either.

In some embodiments, the pharmaceutical composition comprises Compound I and Compound II-d. In some embodiments, the pharmaceutical composition of Compound I and Compound II-d is dosed once daily.

In some embodiments, the CFTR residual function mutation is selected from E56K, P66L, R74W, D110E, D110H, R117C, R 117H, G178R, E193K, L206W, R347H, R352Q, A455E, 5549N, S549R, G551D, G551S, D579G, 711+3A→G, E831X, S9451S977F, F1052V, K1060T, A1067T, R1070W, F10744DI 152H, G1244E, 51251N, S1255P, D1270N, G1349D, 2789+5G→A, 3272-26A→G, and 3849+10kbC→T, 100571 In some embodiments, the residual function mutation is a splice mutation selected. from 2789+5(A, 3272-26A->G, 3849+10kbC4T, 711+3A->G, and E831X, In some embodiments, the splice mutation is E831X.

In some embodiments, the CFTR mutation is E831X.

In some embodiments, the CFTR residual function mutation is a missense mutation selected from D579G, D110H, D1152H, A455E, L206W, P67L, R1W, R117C, R347H, R352Q, 5945L, and 5977F.

In some embodiments, the CFTR residual function mutation is selected from R117H, G178R, S549N, S549R, G551D, G551S, G1244E, S1251N, and G349D.

In one embodiment, the patient is heterozygous for at least one residual function mutation on one allele and a second CFTR gene mutation on the other allele. In another embodiment, the patient is heterozygous for a E831X mutation on one allele and a F508del mutation on the other allele. In one embodiment, the patient has at least one E831X mutation.

It will be appreciated that the disclosed method of modulating a CFTR-mediated disease, particularly cystic fibrosis, in a patient with a residual function mutation, involves a combination therapy. For example, a composition comprising Compound I or a pharmaceutically acceptable salt thereof may be administered in the methods of the invention concurrently with, prior to, or subsequent to a composition comprising Compound II or II-d or a pharmaceutically acceptable salt thereof. In other embodiments, a composition comprising Compound I or a pharmaceutically acceptable salt thereof and a composition comprising Compound II or II-d or a pharmaceutically acceptable salt thereof may be administered. concurrently with, prior to, or subsequent to a composition comprising at least one additional active pharmaceutical ingredient. Alternatively, a single composition comprising Compound I and Compound II or II-d or a pharmaceutically acceptable salt of Compound I or Compound II or II-d or both Compound I and Compound II or II-d, may be administered concurrently with, prior to, or subsequent to a composition comprising at least one additional active pharmaceutical ingredient.

In one aspect, the method of modulating a CFTR-mediated disease in a patient with a residual function mutation involves treating, lessening the severity of, or symptomatically treating cystic fibrosis in the patient by administering an effective amount of Compound I and Compound II or II-d to the patient. In some embodiments, the patient is a mammal.

In certain embodiments, the methods of the invention are useful for treating, lessening the severity of, or symptomatically treating cystic fibrosis in patients who exhibit residual CFTR activity in the apical membrane of respiratory and non-respiratory epithelia. The presence of residual CFTR activity at the epithelial surface can be readily detected using methods known in the art, e.g., standard electrophysiological, biochemical, or histochemical techniques. Such methods identify CFTR activity using in vivo or ex vivo electrophysiological techniques, measurement of sweat or salivary concentrations, or ex vivo biochemical or histochemical techniques to monitor cell surface density of CFTR protein. Using such methods, residual CFTR. activity can be readily detected in patients heterozygous or homozygous for a variety of different mutations, including patients homozygous or heterozygous for the most common mutation, F508del. In certain embodiments, the methods of the invention are useful for treating, lessening the severity of, or symptomatically treating cystic fibrosis in patients who exhibit residual CFTR activity. In certain embodiments, the methods of the invention are useful for treating, lessening the severity of, or symptomatically treating cystic fibrosis in patients who exhibit little to no residual CFTR activity in the apical membrane of respiratory epithelia.

In another embodiment, the compositions disclosed herein are useful for treating or lessening the severity of cystic fibrosis in patients who exhibit residual CFTR activity using pharmacological methods. In another embodiment, the compositions disclosed herein are useful for treating or lessening the severity of cystic fibrosis in patients who have residual CFTR activity using gene therapy. Such methods increase the amount of CFTR present at the cell surface, thereby inducing a hitherto absent CFTR activity in a patient or augmenting the existing level of residual CFTR activity in a patient.

In certain embodiments, the methods of the invention are useful for treating, lessening the severity of, or symptomatically treating cystic fibrosis in patients within certain clinical phenotypes, e.g., a moderate to mild clinical phenotype that typically correlates with the amount of residual CFTR activity in the apical membrane of epithelia. Such phenotypes include patients exhibiting pancreatic sufficiency.

In one embodiment, the compositions disclosed herein are useful for treating, lessening the severity of, or symptomatically treating patients diagnosed with pancreatic sufficiency, idiopathic pancreatitis and congenital bilateral absence of the vas deferens, or mild lung disease, wherein the patient exhibits residual CFTR activity.

The exact amount of a pharmaceutical composition(s) comprising Compound I and Compound II or II-d required in the methods of the invention will vary from subject to subject, depending on the species, age, and general condition of the subject, the severity of the infection, the particular agent, its mode of administration, and the like. Compound I and Compound II or may be formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of this disclosure will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, genetic profile, sex, and diet of the patient; the time of administration, route of administration, and rate of excretion of the specific compound employed; the duration of the treatment; drugs used in combination or coincidental with the specific compound employed, and like factors well known in the medical arts. The term “patient,” as used herein, means an animal, such as a mammal, and even further such as a human.

### Clinical Data

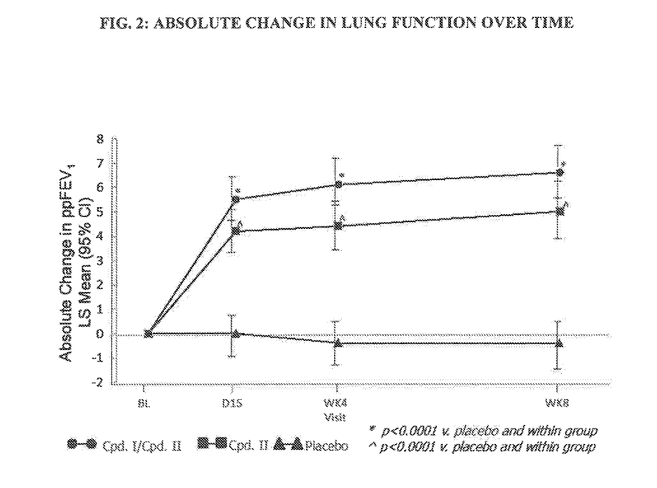

A clinical trial was conducted with 248 cystic fibrosis patients with splice and missense mutations. Mutations were selected for the study based on the clinical phenotype (pancreatic sufficiency), biomarker data (sweat chloride), and in vitro responsiveness to tezacaftor/ivacaftor. The patients were assigned to one of three treatment groups: placebo, Compound II alone, and Compound I and Compound II. These clinical trial results demonstrated that a combination treatment with Compound I and Compound II provides a statistically significant unexpectedly superior improvement in percent predicted forced expiratory volume in one second (ppFEV₁) as compared to the administration of Compound II alone in the treatment of residual function mutations. As shown in. FIG. 2, patients dosed with 100 mg Compound I every 24 hours and 150 mg Compound II every 12 hours displayed an increase in ppFEV₁ of 6.8 percentage points based on the assessment of the average change from baseline after 4 and 8 weeks of treatment, whereas patients dosed with 150 mg Compound II every 12 hours displayed an increase in ppFEV₁ of 4.7 percentage points based on this same assessment. The FEV₁ difference with combination therapy achieved statistically superior results (ΔppFEV₁=2,1 percentage points) over Compound I monotherapy with limited variability (95% CI=1.2,2.9) and consistent response across all pre-specified subgroups.

The patients dosed with 100 mg Compound I every 24 hours and 150 mg Compound II every 12 hours displayed a statistically significant increase in CFQ-R score of 11.1 points after 8 weeks of treatment, as compared to patients dosed with 150 mg Compound II every 12 hours, which resulted in a numerically smaller increase in CFQ-R score of 9.7 points after 8 weeks of treatment.

A second clinical trial was conducted with 150 cystic fibrosis patients aged 12 years and older Who were heterozygous for the F508del mutation and a CFTR residual function mutation. This trial was an eight-week, randomized, double-blind, ivacaftor-controlled, parallel-group study in CF patients. Patients were randomized 1:1 to receive tezacaftor/ivacaftor or ivacaftor following a four-week run in of ivacaftor. The mean ppFEV₁ at baseline was 64.3%. The treatment difference between tezacaftor/ivacaftor and ivacaftor-treated patients for absolute change in ppFEV₁ (primary endpoint) through Week 8 in the active comparator treatment period was 0.3 percentage points.

Results for tezacaftor/ivacaftor- and ivacaftor-treated patients were similar for absolute change in ppFEV₁, relative change in ppFEV₁ and CFQ-R Respiratory Domain Score. In the active comparator treatment period, the mean absolute change in ppFEV ₁ was 0.5% in the tezacaftor/ivacaftor group and 0.2% in the ivacaftor group; the mean relative change in ppFEV₁ was 1.3% in the tezacaftor/ivacaftor group and 0.5% in the ivacaftor group; and the mean absolute change in the pooled CFQ-R respiratory domain score was 0.7 points in the tezacaftor/ivacaftor group and −2.1 points in the ivacaftor group. There was a reduction in sweat chloride in tezacaftor/ivacaftor-treated patients compared to the ivacaftor group (−5.8 mmol/L).

### Methods of Preparing Compound I and Compound II

### General Experimental Procedures

Reagents and starting materials were obtained by commercial sources unless otherwise stated and were used without purification. Proton and carbon NMR spectra were acquired on either of a Bruker Biospin DRX 400 MHz FTNMR spectrometer operating at a and ¹H and ¹³C resonant frequency of 400 and 100 MHz respectively, or on a 300 MHz NMR spectrometer. One dimensional proton and carbon spectra were acquired using a broadband observe (BBFO) probe with 20 Hz sample rotation at 0.1834 and 0.9083 Hz/Pt digital resolution respectively. All proton and carbon spectra were acquired with temperature control at 30° C. using standard, previously published pulse sequences and routine processing parameters. Final purity of compounds was determined by reversed phase UPLC using an Acquity UPLC BEH C₁₈ column (50×2.1 mm, 1.7 μm particle) made by Waters (pn: 186002350), and a dual gradient run from 1-99% mobile phase B over 3.0 minutes. Mobile phase A=H₂O(0.05% CF₃CO₂H). Mobile phase B=CH₃CN (0.035% CF₃CO₂H). Flow rate=1.2 mL/min, injection volume=1.5 μL and column temperature=60° C. Final purity was calculated by averaging the area under the curve (AUC) of two UV traces (220 nm, 254 nm). Low-resolution mass spectra were obtained using a single quadrupole mass spectrometer with a mass accuracy of 0.1 Da and a minimum resolution of 1000 amu across the detection range using electrospray ionization (ESI) using the hydrogen ion (H⁺).

### EXAMPLE 1

### Synthesis of Compound I—(R)-1-(2,2-Difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

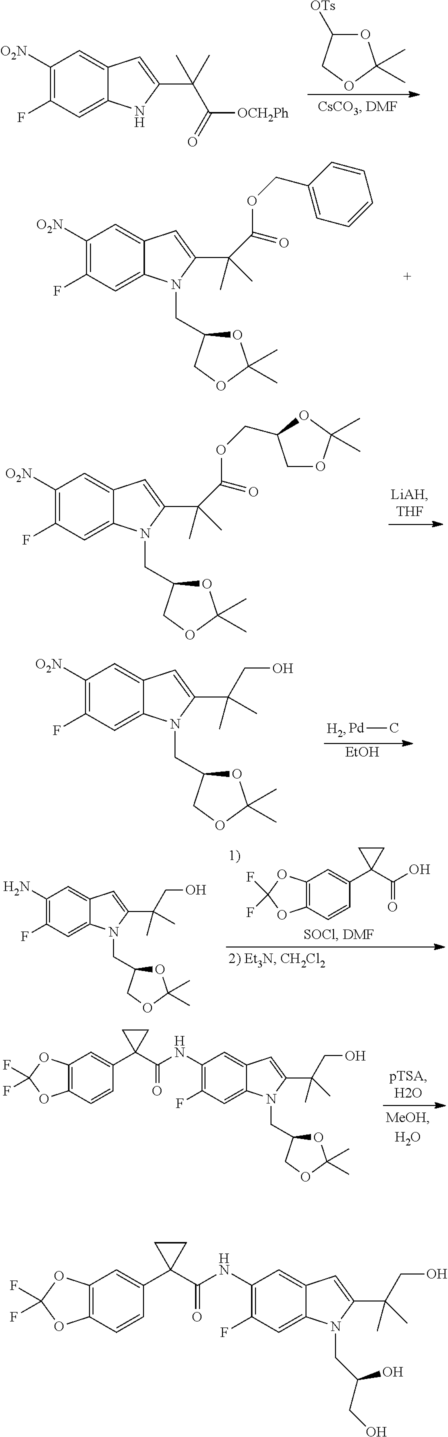

### Step A: (R)-Benzyl 2-(1-((2,2-dimethyl-1,3-dioxolan-4-yl)methyl)-6-fluoro-5-nitro-1H-1-indol-2-yl)-2-methylpropanoate and ((S)-2,2-Dimethyl-1,3-dioxolan-4-yl)methyl 2-(1-(((R)-2,2-dimethyl-1,3-dioxolan-4-yl)methyl)-6-fluoro-5-nitro-1H-indol-2-yl)-2-methylpropanoate

Cesium carbonate (8.23 g, 25.3 mmol) was added to a mixture of benzyl 2-(6-fluoro-5-nitro-1H-indol-2-yl)-2-methylpropanoate (3.0 g, 8.4 mmol) and (S)-(2,2-dimethyl-1,3-dioxolan-4-yl)methyl 4-methylbenzenesulfonate (7.23 g, 25.3 mmol) in DMF (17 mL). The reaction was stirred at 80° C. for 46 hours under a nitrogen atmosphere. The mixture was then partitioned between ethyl acetate and water. The aqueous layer was extracted with ethyl acetate. The combined ethyl acetate layers were washed with brine, dried over MgSO₄, filtered and concentrated. The crude product, a viscous brown oil which contains both of the products shown above, was taken directly to the next step without further purification. (R)-Benzyl 2-(1-((2,2-dimethyl-1,3-dioxolan-4-yl)methyl)-6-fluoro-5-nitro-1H-indol-2-yl)-2-methylpropanoate, ESI-MS m/z calc. 470.2, found 471.5 (M+1)⁻. Retention time 2.20 minutes. ((S)-2,2-Dimethyl-1,3-dioxolan-4-yl)methyl 2-(1 -(((R)-2,2-dimethyl-1,3-dioxolan-4-yl)methyl)-6-fluoro-5-nitro-1H-indol-2-yl)-2-methylpropanoate, ESI-MS m/z calc. 494.5, found 495.7 (M+1)⁺. Retention time 2.01 minutes.

### Step B: (R)-2-(1-((2,2-dimethyl-1,3-dioxolan-4-yl)methyl)-6-fluoro-5-nitro-1H-indol-2-yl)-2-methylpropan-1-ol

The crude reaction mixture obtained in step (A) was dissolved in THF (42 mL) and cooled in an ice-water bath. LiAlH₄ (16.8 mL of 1 M solution, 16.8 mmol) was added drop-wise. After the addition was complete, the mixture was stirred for an additional 5 minutes. The reaction was quenched by adding water (1 mL), 15% NaOH solution (1 mL) and then water (3 mL). The mixture was filtered over Celite, and the solids were washed with THF and ethyl acetate. The filtrate was concentrated and purified by column chromatography (30-60% ethyl acetate-hexanes) to obtain (R)-2-(1-((2,2-dimethyl-1,3-dioxolan-4-yl)methyl)-6-fluoro-5-nitro-1H-indol-2-yl)-2-methylpropan-1-ol as a brown oil (2.68 g, 87% over 2 steps). ESI-MS m/z calc. 366.4, found 367.3 (M+1)⁺. Retention time 1.68 minutes. ¹H NMR (400 MHz, DMSO-d6) δ 8.34 (d, J=7.6 Hz, 1H), 7.65 (d, J=13.4 Hz, 1H), 6.57 (s, 1H), 4.94 (t, J=5.4 Hz, 1H), 4.64-4.60(m, 1H), 4.52-4.42(m, 2H), 4.16-4.14 (m, 1H), 3.76-3.74 (m, 1H), 3.63-3.53 (m, 2H), 1.42. (s, 3H), 1.38-1.36 (m, 6H) and 1.19 (s, 3H) ppm.

### Step C: (R)-2-(5-amino-1-((2,2-dimethyl-1,3-dioxolan-4-yl)methyl)-6-fluoro-1H-indol-2-yl)-2-methylpropan-1-ol

(R)-2-(1-(2,2-dimethyl-1,3-dioxolan-4-yl)methyl)-6-fluoro-5-nitro-1H-indol-2-yl)-2-methylpropan-1-ol (2.5 g, 6.82 mmol) was dissolved in ethanol (70 mL) and the reaction was flushed with N₂. Then Pd—C (250 mg, 5% wt) was added. The reaction was flushed with nitrogen again and then stirred under H₂ (atm). After 2.5 hours only partial conversion to the product was observed by LCMS. The reaction was filtered through Celite and concentrated. The residue was re-subjected to the conditions above. After 2 hours LCMS indicated complete conversion to product. The reaction mixture was filtered through Celite. The filtrate was concentrated to yield the product as a black solid (1.82 g, 79%). ESI-MS m/z calc. 336.2, found 337.5 (M+1)⁺. Retention time 0.86 minutes. ¹H NMR (400 MHz, DMSO-d6) δ 7.17 (d, J=12.6 Hz, 1H), 6.76 (d, J=9.0 Hz, 1H), 6.03 (s, 1H), 4.79-4.76 (m, 1H), 4.46 (s, 2H), 4.37-4.31 (m, 3H),4,06 (dd, J=6.1, 8.3 Hz, 1H), 3.70-3.67 (m, 1H), 3.55-3.52 (m, 2H), 1.41 (s, 3H), 1.32 (s, 6H) and 1.21 (s, 3H) ppm.

### Step D: (R)-1-(2,2-difluorobenzo[d][1,3]dioxol-5-yl)-N-(1-((2,2-dimethyl-1,3-dioxiolan-4-yl)methyl)-6-fluoro-2-(1-hydroxy-2-methylpropan-2-yl)-1H-indol-5-yl)cyclopropanecarboxamide

DMF (3 drops) was added to a stirring mixture of 1-(2,2-difluorobenzo[d][1,3]dioxol-5-yl)cyclopropanecarboxylic acid (1.87 g, 7.7 mmol) and thionyl chloride (1.30 mL, 17.9 mmol); After 1 hour a clear solution had formed. The solution was concentrated under vacuum and then toluene (3 mL) was added and the mixture was concentrated again. The toluene step was repeated once more and the residue was placed on high vacuum for 10 minutes. The acid chloride was then dissolved in dichloromethane (10 mL) and added to a mixture of (R)-2-(5-amino-1-((2,2-dimethyl-1,3 -dioxolan-4-yl)methyl)-6-fluoro-1H-indol-2-yl)-2-methylpropan-1-ol (1.8 g, 5.4 mmol) and triethylamine (2.24 mL, 16.1 mmol) in dichloromethane (45 mL). The reaction was stirred at room temperature for 1 hour. The reaction was washed with 1N HCl solution, saturated NaHCO₃ solution and brine, dried over MgSO₄ and concentrated to yield the product as a black foamy solid (3g, 100%). ESI-MS m/z calc. 560.6, found 561.7 (M×1)⁺. Retention time 2.05 minutes. ¹H NMR (400 MHz, DMSO-d6) δ 8.31 (s, 1H), 7.53 s, 1H), 7.42-7.40 (m, 2H), 7.34-7.30 (m, 3H), 6.24 (s, 1H), 4.51-4.48 (m, 1H), 4.39-4.34 (in,2H), 4.08 (dd, J=6.0, 8.3 Hz, 1H), 3.69 (t, J=7.6 Hz, 1H), 3.58-3.51 (m, 2H), 1.48-1.45 (m, 2H), 1.39 (s, 3H), 1.34-1.33 (m, 6H), 1.18 (s, 3H) and 1.14-1.12(m, 2H) ppm.

### Step E: (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide

(R)-1-(2,2-difluorobenzo[d][1,3]dioxol-5-yl)-N-(1-((2,2-dimethyl-1,3-dioxolan-4-yl)methyl)-6-fluoro-2-(1-hydroxy-2-methylpropan-2-yl)-1H-indol-5-yl)cyclopropanecarboxamide (3.0 g, 5.4 mmol) was dissolved in methanol (52 mL). Water (5.2 mL) was added followed by p-TsOH.H₂O (204 mg, 1.1 mmol). The reaction was heated at 80° C. for 45 minutes. The solution was concentrated and then partitioned between ethyl acetate and saturated NaHCO₃ solution. The ethyl acetate layer was dried over MgSO₄ and concentrated. The residue was purified by column chromatography (50-100% ethyl acetate hexanes) to yield the product as a cream colored foamy solid (1.3 g, 47%, ee>98% by SFC). ESI-MS m/z calc. 520.5, found 521.7 (M+1)⁺. Retention time 1.69 minutes. ¹NMR (400 MHz, DMSO-d6) δ 8.31 (s, 1H), 7.53 (s, 1H), 7.42-7.38 (m, 2H), 7.33-7.30 (m, 2H), 6.22 (s, 1H), 5.01 (d, J=5.2 Hz, 1H), 4.90 (t, J=5.5 Hz, 1H), 4.75 (t, J=5.8 Hz, 1H), 4.40 (dd, J=2.6, 15.1 Hz, 1H), 4.10 (dd, J=8.7, 15.1 Hz, 1H), 3.90 (s, 1H), 3.65-3.54 (m, 2H), 3.48-3.33 (m, 2H), 1.48-1.45 (m, 2H), 1.35 (s, 3H), 1.32 (s, 3H) and 1.14-1.11 (m, 2H) ppm.

### Example 2

### Synthesis of Compound II—N-(2,4-di-tert-butyl-5-hydroxyphenyl)-4-oxo-1,4-dihydroquinoline-3-carboxamide

### Part A: Preparation of 4-oxo-1,4-dihydroquinoline-3-carboxylic acid

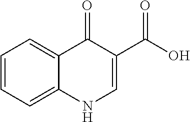 [SMILES: O=C(O)c1c[nH]c2ccccc2c1=O]

### Step A: 2-Phenylaminomethylene-malonic acid diethyl ester

A mixture of aniline (25.6 g, 0.275 mol) and diethyl 2-(ethoxymethylene)malonate (62.4 g, 0.288 mol) was heated at 140-150 C. for 2 h. The mixture was cooled to room temperature and dried under reduced pressure to afford 2-phenylaminomethylene-malonic acid diethyl ester as a solid, which was used in the next step without further purification. ¹H NMR (DMSO-d₆) δ 11.00 (d, 1H), 8.54 (d, J=13.6 Hz, 1H), 7.36-7.39 (m, 2H), 7.13-7.17 (m, 3H), 4.17-4.33 (m, 4H), 1.18-1.40 (m, 6H) ppm.

### Step B: 4-Hydroxyquinoline-3-carboxylic acid ethyl ester

A 1 L three-necked flask fitted with a mechanical stirrer was charged with 2-phenylaminomethylene-malonic acid diethyl ester (26.3 g, 0.100 mol), polyphosphoric acid (270 g) and phosphoryl chloride (750 g). The mixture was heated to 70° C. and stirred for 4 h. The mixture was cooled to room temperature and filtered. The residue was treated with aqueous Na₂CO₃ solution, filtered, washed with water and dried. 4-Hydroxyquinoline-3-carboxylic acid ethyl ester was obtained as a pale brown solid (15.2 g, 70%). The crude product was used in the next step without further purification.

### Step C: 4-Oxo-1,4-dihydroquinoline-3-carboxylic acid

4-Hydroxyquinoline-3-carboxylic acid ethyl ester (15 g, 69 mmol) was suspended in a sodium hydroxide solution (2N, 150 mL) and stirred for 2 h at reflux. After cooling, the mixture was filtered, and the filtrate was acidified to pH 4 with 2N HCl. The resulting precipitate was collected via filtration, washed with water and dried under vacuum to give 4-oxo-1,4-dihydroquinoline-3-carboxylic acid as a pale white solid (10.5 g, 92%). ¹H NMR (DMSO-d₆) δ 15.34 (s, 1H), 13.42 (s, 1H), 8.89 (s, 1H), 8.28 (d, J=8.0 Hz, 1H), 7.88 (m, 1H), 7.81 (d, J=8.4 Hz, 1H), 7.60 (m, 1H) ppm.

### Part B: N-(2,4-di-tert-butyl-5-hydroxyphenyl)-4-oxo-1,4-dihydroquinoline-3-carboxamide

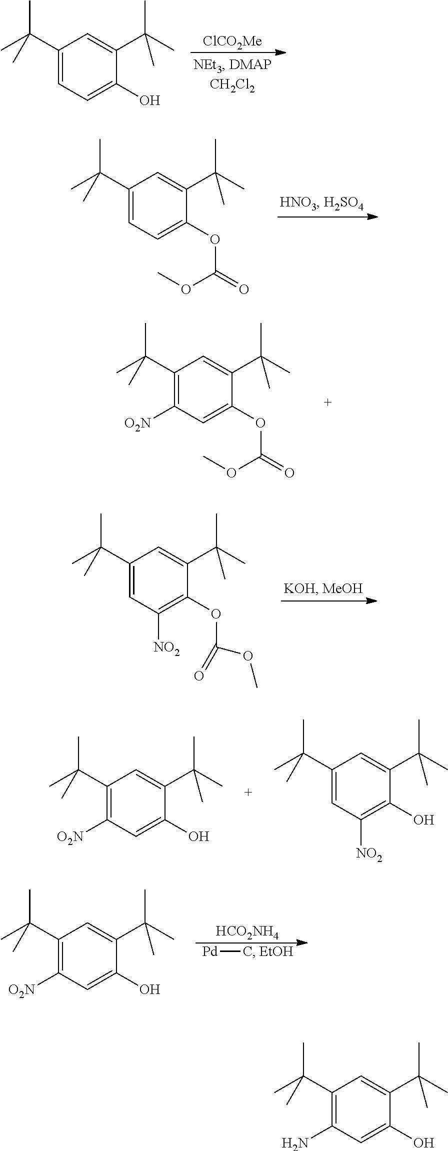

### Step A: Carbonic acid 2,4di-tert-butyl-phenyl ester methyl ester

Methyl chloroformate (58 mL, 750 mmol) was added dropwise to a solution of 2,4-di-tert-butyl-phenol (103.2 g, 500 mmol), Et₃N (139 mL, 1000 mmol) and DMAP (3.05 g, 25 mmol) in dichloromethane (400 mL) cooled in an ice-water bath to 0° C. The mixture was allowed to warm to room temperature while stirring overnight, then filtered through silica gel (approx. 1L) using 10% ethyl acetate-hexanes (˜4 L) as the eluent. The combined filtrates were concentrated to yield carbonic acid 2,4-di-tert-butyl-phenyl ester methyl ester as a yellow oil (132 g, quant.), ¹ NMR (400 MHz, DMSO-d₆) δ 7.35 (d, J=2.4 Hz, 1H), 7.29 (dd, J=8.5, 2.4 Hz, 1H), 7.06 (d, J=8.4 Hz, 1H), 3.85 (s, 3H), 1.30 (s, 9H), 1.29 (s, 9H) ppm.

### Step B: Carbonic acid 2,4-di-tert-butyl-5-nitro-phenyl ester methyl ester and Carbonic acid 2,4-di-tert-butyl-6-nitro-phenyl ester methyl ester

To a stirring mixture of carbonic acid 2,4-di-tert-butyl-phenyl ester methyl ester (4.76 g, 180 mmol) in conc. sulfuric acid (2 mL), cooled in an ice-water bath, was added a cooled mixture of sulfuric acid (2 mL) and nitric acid (2 mL). The addition was done slowly so that the reaction temperature did not exceed 50° C. The reaction was allowed to stir for 2 h while warming to room temperature. The reaction mixture was then added to ice-water and extracted into diethyl ether. The ether layer was dried (MgSO₄), concentrated and purified by column chromatography (0-10% ethyl acetate hexanes) to yield a mixture of carbonic acid 2,4-di-tert-butyl-5-nitro-phenyl ester methyl ester and carbonic acid 2,4-di-tert-butyl-6-nitro-phenyl ester methyl ester as a pale yellow solid (4.28 g), which was used directly in the next step.

### Step C: 2,4-Di-tert-butyl-5-nitro-phenol and 2,4-Di-tert-butyl-6-nitro-phenol

The mixture of carbonic acid 2,4-di-tert-butyl-5-nitro-phenyl ester methyl ester and carbonic acid 2,4-di-tert-butyl-6-nitro-phenyl ester methyl ester (4.2 g, 14.0 mmol) was dissolved in MeOH (65 mL) before KOH (2.0 g, 36 mmol) was added. The mixture was stirred at room temperature for 2 h. The reaction mixture was then made acidic (pH 2-3) by adding cone. HCl and partitioned between water and diethyl ether. The ether layer was dried (MgSO₄), concentrated and purified by column chromatography (0-5% ethyl acetate-hexanes) to provide 2,4-di-tert-butyl-5-nitro-phenol (1.31 g, 29% over 2 steps) and 2,4-di-tert-butyl-6-nitro-phenol. 2,4-Di-tert-butyl-5-nitro-phenol: ¹H NMR (400 MHz, DMSO-d6) δ 10.14 (s, 1H, OH), 7.34 (s, 1H), 6.83 (s, 1H), 1.36 (s, 9H), 1.30 (s, 9H) ppm. 2,4-Di-tert-butyl-6-nitro-phenol: ¹H NMR (400 MHz, CDCl₃) δ 11.48 (s, 1H), 7.98 (d, J=2.5 Hz, 1H), 7.66 (d, J=2.4 Hz, 1H), 1.47 (s, 9H), 1.34 (s, 9H) ppm.

### Step D: 5-Amino-2,4-di-tert-butyl-phenol

To a refluxing solution of 2,4-di-tert-butyl-5-nitro-phenol (1.86 g, 7.40 mmol) and ammonium formate (1.86 g) in ethanol (75 mL) was added Pd-5% wt. on activated carbon (900 mg). The reaction mixture was stirred at reflux for 2 h, cooled to room temperature and filtered through Celite. The Celite was washed with methanol and the combined filtrates were concentrated to yield 5-amino-2,4-di-tert-butyl-phenol as a grey solid (1.66 g, quant.). ¹H NMR (400 MHz, DMSO-d₆) δ 8.64 (s, 1H, OH), 6.84 (s, 1H), 6.08 (s, 1H), 4.39 (s, 2H, NH₂), 1.27 (m, 18H) ppm; HPLC ret. time 2.72 min, 10-99% CH₃CN, 5 min run; ESI-MS 222.4 m/z [M+H]⁺.

### Step E: N-(5-hydroxy-2,4-di-tert-butyl-phenyl)-4-oxo-1H-quinoline-3-carboxamide

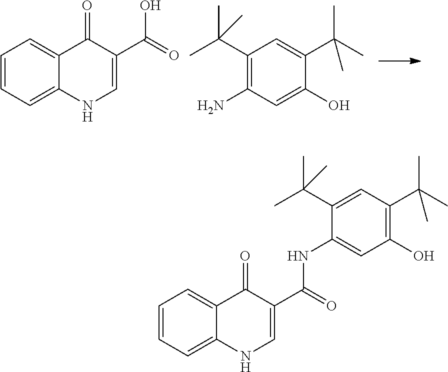

To a suspension of 4-oxo-1,4-dihydroquinolin-3-carboxylic acid (35.5 g, 188 mmol) and HBTU (85.7 g, 226 mmol) in DMF (2.80 mL) was added Et₃N (63.0 mL, 451 mmol) at ambient temperature. The mixture became homogeneous and was allowed to stir for 10 min before 5-amino-2,4-di-tert-butyl-phenol (50.0 g, 226 mmol) was added in small portions. The mixture was allowed to stir overnight at ambient temperature. The mixture became heterogeneous over the course of the reaction. After all of the acid was consumed (LC-MS analysis, MH+ 190, 1.71 min), the solvent was removed in vacuo. EtOH was added to the orange solid material to produce a slurry. The mixture was stirred on a rotovap (bath temperature 65° C.) for 15 min without placing the system under vacuum. The mixture was filtered and the captured solid was washed with hexanes to provide a white solid that was the EtOH crystalate. Et₂O was added to the solid obtained above until a slurry was formed. The mixture was stirred on a rotovapor (bath temperature 25° C.) for 15 min without placing the system under vacuum. The mixture was filtered and the solid captured. This procedure was performed a total of five times. The solid obtained after the fifth precipitation was placed under vacuum overnight to provide 1-(5-hydroxy-2,4-di-tert-butyl-phenyl)-4-oxo-1H-quinoline-3-carboxamide as a white powdery solid (38 g, 52%). HPLC ret. time 3.45 min, 10-99% CH₃CN, 5 min run; ¹H NMR (400 MHz, DMSO-d₆) δ 12.88 (s, 1H), 11.83 (s, 1H), 9.20 (s, 1H), 8.87 (s, 1H), 8.33 (dd, J=8.2, 1.0 Hz, 1H), 7.83-7.79 (m. 1H), 7.76 (d, J=7.7 Hz, 1H), 7.54-7.50 (m, 1H), 7.17 (s, 1H), 7.10 (s, 1H), 1.38 (s, 9H), 1.37 (s, 9H) ppm; ESI-MS m/z calc'd 392.21; toured 393.3 [M+H]⁺.

### EXAMPLE 3

### Preparation of a Solid Dispersion Comprising Substantially Amorphous Compound I and HPMC Polymer

A solvent system of dichloromethane (DCM) and methanol (MeOH), was formulated according to the ratio 80 wt % DCM/20 wt % MeOH, in an appropriately sized container, equipped with a magnetic stirrer and stir plate. Into this solvent system, hypromellose polymer (HPMC, E15 grade) and Compound I were added according to the ratio 20 wt % hypromellose/80 wt % Compound I. The resulting mixture contained 12.5 wt % solids. The actual amounts of ingredients and solvents used to generate this mixture are recited in Table 2, below:

TABLE 2
Solid spray dispersion ingredients for amorphous Compound I

| | Units | Batch |
|---|---|---|
| Compound I | g | 2400 |
| HPMC | g | 600 |
| Total Solids | g | 3000 |
| DCM | g | 16800 |
| MeOH | g | 4200 |
| Total Solvents | g | 21000 |
| Total Spray Solution Weight | g | 24000 |

The mixture was mixed until it was substantially homogenous and all components were substantially dissolved.

A spray drier, Anhydro MS-35 Spray Drier, fitted with two fluid 0.8 mm nozzle (Schlick series 970/0 S4), was used under normal spray drying mode, following the dry spray process parameters recited in Table 3, below.

TABLE 3
Spray drying dispersion processing parameters to generate solid spray
dispersion of amorphous Compound I

| Parameter | Value | |
|---|---|---|
| Process Gas Flow Rate | 34 | Kg/hr |
| Nozzle Gas Flow Rate | 4.2 | Kg/hr |
| Feed Flow Rate | 2 | Kg/hr |
| Inlet Temperature | 96-108° | C. |
| Outlet Temperature | 40° | C. |
| Vacuum Dryer Temperature | 45° | C. |
| Vacuum Drying Time | 24-72 | hours |

A high efficiency cyclone separated the wet product from the spray gas and solvent vapors. The wet product was transferred into trays and placed in a vacuum dryer for drying to reduce residual solvents to a level of less than about 3000 ppm for MeOH and less than 600 ppm of DCM and to generate dry spray dry dispersion of amorphous Compound I, containing <0.02% MeOH and <0.06% DCM.

### EXAMPLE 4

### Preparation of a Solid Dispersion Comprising Substantially Amorphous Compound II and HPMCAS Polymer

A solvent system of MEK and DI water, formulated according to the ratio 90 wt % MEK/10 wt % DI water, was heated to a temperature of 20-30° C. in a reactor, equipped with a magnetic stirrer and thermal circuit. Into this solvent system, hypromellose acetate succinate polymer (HPMCAS)(HG grade), SLS, and Compound II were added according to the ratio 19,5 wt % hypromellose acetate succinate/0.5 wt % SLS/80 wt % Compound II. The resulting mixture contained 10.5 wt % solids. The actual amounts of ingredients and solvents used to generate this mixture are recited in Table 4, below.

TABLE 4
Solid spray dispersion ingredients for amorphous compound II.

| | |
|---|---|
| Units | Batch |

| | | |
|---|---|---|
| Compound 2 | Kg | 70.0 |
| HPMCAS | Kg | 17.1 |
| SLS | Kg | 0.438 |
| Total Solids | Kg | 87.5 |
| MEK | Kg | 671 |
| Water | Kg | 74.6 |
| Total Solvents | Kg | 746 |
| Total Spray Solution Weight | Kg | 833 |

The mixture temperature was adjusted to a range of 20-45° C. and mixed until it was substantially homogenous and all components were substantially dissolved.

A spray drier, Niro PSD4 Commercial Spray Dryer, fitted with pressure nozzle (Spray Systems Maximum Passage series SK-MFP having orifice/core size 54/21) equipped with anti-bearding cap, was used under normal spray drying mode, following the dry spray process parameters recited in Table 5, below.

TABLE 5
Spray drying dispersion processing parameters to generate solid spray
dispersion of amorphous Compound II.

| Parameter: | Value: | |
|---|---|---|
| Feed Pressure | 20 | bar |
| Feed Flow Rate | 92-100 | Kg/hr |
| Inlet Temperature | 93-99° | C. |
| Outlet Temperature | 53-57° | C. |
| Vacuum Dryer Temperature | 80° C. for 2 hours then | |
| | 110° C. (+/−5° C.) | |
| Vacuum Drying Time | 20-24 | hours |

A high efficiency cyclone separated the wet product from the spray gas and solvent vapors. The wet product contained 8.5-9.7% MEK and 0.56-0.83% water and had a mean particle size of 17-19 μm and a bulk density of 0.27-0.33 g/cc. The wet product was transferred to a 4000 L stainless steel double cone vacuum dryer for drying to reduce residual solvents to a level of less than about 5000 ppm and to generate dry spray dry dispersion of amorphous Compound 11, containing <0.03% MEK and 0.3% water.

### EXAMPLE 5

### Preparation of a 100 mg Compound I and 150 mg Compound II Tablet Formation from Dry Granulation Roller Compaction

Equipment:

Turbula blender, V-shell blender or a bin blender, Gerteis Roller Compactor, Courtoy tablet press, Omega coating system.

Screening/Weighing:

The solid dispersion comprising substantially amorphous Compound I, the solid dispersion comprising substantially amorphous Compound II, and excipients may be screened prior to or after weigh-out. Appropriate screen sizes are 24R, or mesh 60.

Blending:

The solid dispersion comprising substantially amorphous Compound I, the solid dispersion comprising substantially amorphous Compound II, and excipients may be added to the blender in different order. The blending may be performed in a Turbula blender, a v-shell blender, or a bin blender. The components may be blended for 25 minutes.

Dry Granulation:

The blend may be granulated using a Gerteis roller compactor. The blend may be granulated using combined smooth/smooth rolls and with the integrated 0.8 mm mesh milling screen with pocketed rotor and paddle agitator. The Gerteis roller compactor may be operated with a roll gap of 3 mm, roll pressure of 10 kNcm, roll speed of 8 rpm, agitator speed 15 rpm, granulation speed clockwise/counterclockwise of 150/150 rpm, and oscillation clockwise/counterclockwise of 375/375 degrees. The ribbons produced may be milled with integrated mill equipped with 0.8 mm mesh screen.

Blending:

The roller compacted granules may be blended with extra-granular excipients such as filler and, if needed lubricant using a Turbula blender, V-shell blender or a bin blender. The blending time may be 7 minutes or may be lubed for 5 minutes.

Compression:

The compression blend may be compressed into tablets using a single station or rotary tablet presses, such as the Courtoy tablet press, using Tooling Size D Caplet Tooling (0.625 ″×0.334″). The weight of the tablets for a dose of 100 mg of substantially amorphous Compound I and 150 mg of substantially amorphous Compound II may be about 500 to 700 mg.

Coating

The core tablets are film coated using a continuous pan Omega coater. The film coat suspension is prepared by adding the Opadry yellow 20A120010 powder to purified water. The required amount of film coating suspension (3% of the tablet weight) is sprayed onto the tablets to achieve the desired weight gain.

TABLE 6
Tablet Comprising 100 mg Compound I and 150 mg Compound II.

| | | Amount per tablet |
|---|---|---|
| | Ingredient | mg |
| Intra-granular | Compound I SDD | 125 |
| | Compound II SDD | 187.5 |
| | Microcrystalline cellulose | 131.4 |
| | Croscarmellose Sodium | 29.6 |
| | Total | 473.5 |
| Extra-granular | Microcrystalline cellulose | 112.5 |
| | Magnesium Stearate | 5.9 |
| | Total | 118.4 |
| Total uncoated | | 591.9 |
| Tablet | | |
| Film coat | Opadry | 17.7 |
| Total coated | | 609.6 |
| Tablet | | |

The foregoing discussion discloses and describes merely exemplary embodiments of this disclosure. One skilled in the art will readily recognize from such discussion and from the accompanying drawings and claims, that various changes, modifications and variations can be made therein without departing from the spirit and scope of this disclosure as defined in the following Claims.

1. A method of treating cystic fibrosis in a patient, comprising administering to the patient an effective amount of (R)-1-(2,2-difluorobenzo[d][1,3]dioxol-5-yl)-N-(1-(2,3-dihydroxypropyl)-6-fluoro-2-(1-hydroxy-2-methylpropan-2-yl)-1H-indol-5-yl)cyclopropanecarboxamide (Compound I): 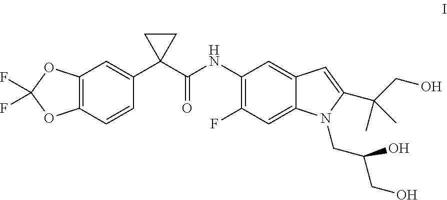 [SMILES: CC(C)(CO)c1cc2cc(NC(=O)C3(c4ccc5c(c4)OC(F)(F)O5)CC3)c(F)cc2n1C[C@@H](O)CO]
or a pharmaceutically acceptable salt thereof and N-(2,4-di-tert-butyl-5-hydroxyphenyl)-4-oxo-1,4-dihydroquinoline-3-carboxamide (Compound II): 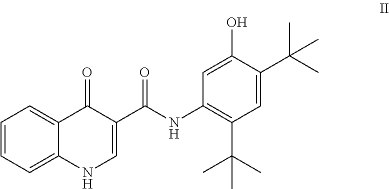 [SMILES: CC(C)(C)c1cc(C(C)(C)C)c(NC(=O)c2c[nH]c3ccccc3c2=O)cc1O]
or N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound II-d): 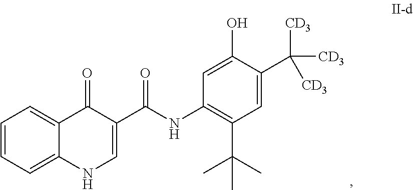 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]
or a pharmaceutically acceptable salt of either,
wherein the patient has at least one E831X cystic fibrosis transmembrane conductance regulator (CFTR) mutation.

2. The method according to claim 1, comprising administering to the patient an effective amount N-(2,4-di-tert-butyl-5-hydroxyphenyl)-4-oxo-1,4-dihydroquinoline-3-carboxamide (Compound II): 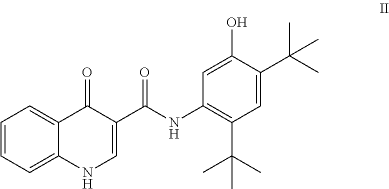 [SMILES: CC(C)(C)c1cc(C(C)(C)C)c(NC(=O)c2c[nH]c3ccccc3c2=O)cc1O]
or a pharmaceutically acceptable salt thereof.

3. The method according to claim 1, comprising administering to the patient an effective amount of N-(2-(tert-butyl)-5-hydroxy-4-(2-(methyl-d3)propan-2-yl-1,1,1,3,3,3-d6)phenyl)-4-oxo-1,4-dihydroquinoline-3-carboxamide (Compound II-d). 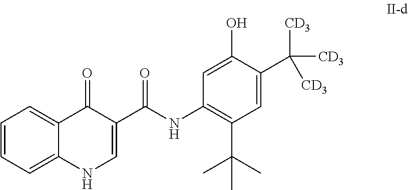 [SMILES: [2H]C([2H])([2H])C(c1cc(C(C)(C)C)c(NC(=O)c2c[nH]c3ccccc3c2=O)cc1O)(C([2H])([2H])[2H])C([2H])([2H])[2H]]
or a pharmaceutically acceptable salt thereof.

4. The method according to claim 1, wherein the patient has a second CFTR mutation that is F508del.

5. The method according to claim 2, wherein the patient has a second CFTR mutation that is F508del.

6. The method according to claim 3, wherein the patient has a second CFTR mutation that is F508del.

7. The method according to claim 1, comprising administering a pharmaceutical composition of Compound I or a pharmaceutically acceptable salt thereof concurrently with, prior to, or subsequent to a pharmaceutical composition comprising Compound II or II-d or a pharmaceutically acceptable salt thereof.

8. The method according to claim 7, further comprising administering a pharmaceutical composition comprising at least one additional active pharmaceutical ingredient.

9. The method according to claim 8, wherein the at least one additional active pharmaceutical ingredient is administered simultaneously, sequentially, in a single composition, or as one or more separate compositions.

10. The method according to claim 8, wherein the at least one additional active pharmaceutical ingredient is a CFTR modulator.

11. The method according to claim 2, further comprising administering a pharmaceutical composition comprising the at least one additional active pharmaceutical ingredient, wherein the at least one additional active pharmaceutical ingredient is a CFTR modulator.

12. The method according to claim 3, further comprising administering a pharmaceutical composition comprising the at least one additional active pharmaceutical ingredient, wherein the at least one additional active pharmaceutical ingredient is a CFTR modulator.

13. The method according to claim 8, wherein the CFTR modulator is selected from a CFTR corrector and a CFTR potentiator.

14. The method according to claim 1, wherein the patient exhibits residual CFTR activity in the apical membrane of respiratory and non-respiratory epithelia.

15. The method according to claim 1, wherein the patient exhibits little to no CFTR activity in the apical membrane of respiratory epithelia.
